(19) United States
(12) Patent Application Publication
(10) Pub. No.: US 2010/0256656 A1
(43) Pub. Date: Oct. 7, 2010

(54) Suture Passing Device with Controllable Suture Retrieval Mechanism

(76) Inventor: SangDo Park, Marina Del Rey, CA (US)

Correspondence Address: SANGDO PARK, 14001 Palawan Way, Apt. 102, Marina del Rey, CA, 90292, US

(21) Appl. No.: 12/728,273

(22) Filed: Mar. 22, 2010

Related U.S. Application Data
(60) Provisional application No. 61/166,732, filed on Apr. 4, 2009.

(51) Int. Cl. A61B 17/04

(52) U.S. Cl. 606/145

(57) ABSTRACT

The current invention is a modification to suture passing devices which employ a configuration of having upper and lower jaws to both grasp a tissue and deploy a suture loaded needle from one of the jaws. The current invention allows for an operator-controlled means to grasp the passed suture with a mechanism located within the jaw opposite the side of needle deployment. A sliding block located within the jaw opposite the side of needle deployment is controllable by the operator near the handle of the device and provides movement of the sliding block against the inner surface of the distal end of the jaw to exert compression on the passed suture. This compression allows for grasping and retrieval of the passed suture.

This application claims the benefit of U.S. Provisional Patent Application No. 61/166,732 filed on Apr. 4, 2009.

### BACKGROUND OF THE INVENTION

1. Technical Field

This device is of relevance to the field of arthroscopic surgery.

2. Description of the Prior Art

There have been several inventions which seek to facilitate the passage of suture through a tissue via arthroscopic means. The most effective and widely used of these inventions share the common configuration of being elongate in appearance and having at one end upper and lower jaws to both grasp tissue and pass a suture loaded needle, which is usually deployed from the lower jaw, and at the other end of the device, a handle with means to control the action of the jaw and passage of the needle. However, to date, there exists no practical and effective mechanism to retrieve the suture once it is passed through the tissue with the very same device. A hook-like configuration on the jaw opposite the jaw with the needle has been commonly mentioned in various inventions to hook onto the passed suture loop in order to retrieve the suture; however, it is often encountered in surgery that the passed suture is not a loop but rather a free end of the suture. In such an instance, the hook configuration is ineffective. Yet another mechanism that has been used is to release the grasped tissue entirely after the suture is passed, relocate the device to the site of the passed suture, and then grasp the passed suture with the jaws of the device. The problem with this approach is that it is sometimes technically difficult to relocate the passed suture once the device disengages from the suture, and it adds time to the procedure. In actuality, the most commonly employed means to retrieve the passed suture is through the creation of an additional opening on the skin through which a second tong like instrument is passed, and the instrument grasps the suture as soon as it is passed through the tissue and pulls it out through the skin. This adds to the operative time and creates an unnecessary skin scar.

The current invention circumvents the difficulty of retrieving the passed suture. It allows for an operator-controlled means to grasp the passed suture with a simple novel mechanism located within the jaw opposite the side of needle deployment.

### BRIEF SUMMARY OF THE INVENTION

The current invention is a modification to suture passing devices which employ a configuration of having upper and lower jaws to both grasp a tissue and deploy a suture loaded needle from one of the jaws. The current invention allows for an operator-controlled means to grasp the passed suture with a mechanism located within the jaw opposite the side of needle deployment. A sliding block located within the jaw opposite the side of needle deployment is controllable by the operator near the handle of the device and its movement against the inner surface of the distal end of the jaw exerts compression on the passed suture. The movement of the sliding block is accomplished by a long rod which is housed within the suture passing apparatus and passes into the jaw with the manual force exerted by the operator at which time it engages one end of the sliding block and slides it distally against the passed suture and the inner surface of the distal end of the jaw. The compression thus achieved allows for grasping and retrieval of the passed suture.

### BRIEF DESCRIPTION OF THE DRAWINGS

For a fuller understanding of the nature and objects of the invention, reference should be had to the following detailed description taken in conjunction with the accompanying drawings in which:

FIG. 1 is a schematic view of the suture passing device

FIG. 2 is a view of the upper jaw as seen from the top and a cross sectional view of the body portion of the suture passing apparatus

FIG. 3 is a view of the upper jaw as seen from the top and a cross sectional view of the body portion of the suture passing apparatus with the sliding block engaged against the inner surface of the distal end of the jaw

FIG. 4 is a view of the upper jaw as seen from the top and a cross sectional view of the body portion of the suture passing apparatus in an alternative embodiment

### DETAILED DESCRIPTION OF THE PREFERRED EMBODIMENTS

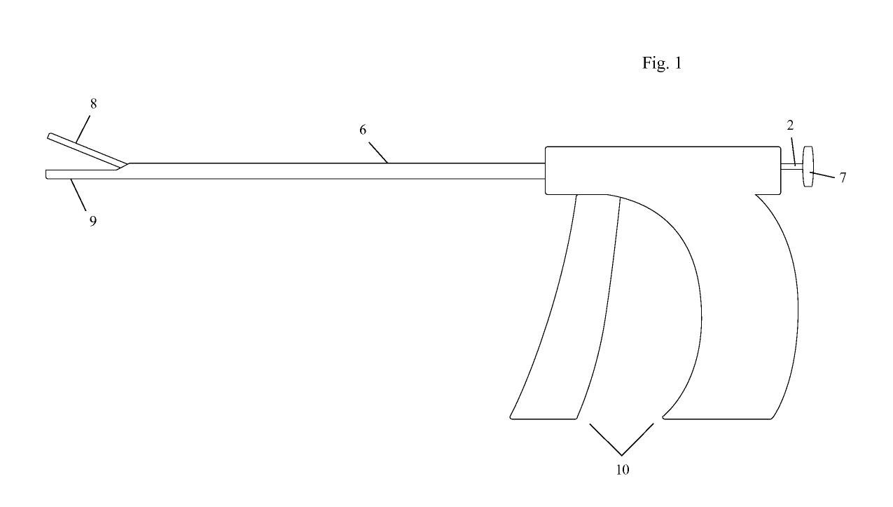

FIG. 1 is a schematic view of the suture passing device. It is comprised of upper jaw 8, lower jaw 9, body portion 6, handle portion 10, and a control member 7. The lower jaw 9 is the side from which deployment of suture loaded needle occurs. The upper jaw 8 is opposite lower jaw 9, and it is the side containing the mechanism for grasping and retrieval of the suture.

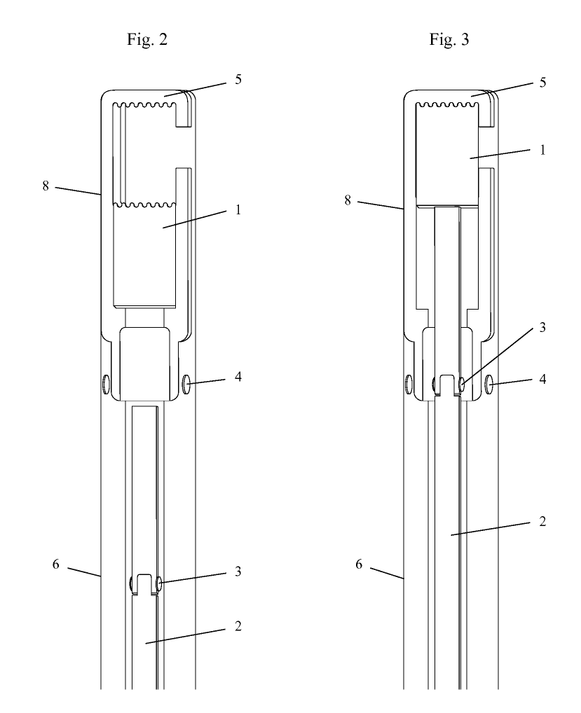

FIG. 2 is a view of the upper jaw 8 as seen from the top and a cross sectional view of the body portion 6 demonstrating sliding block 1 located within the upper jaw 8. A long rod 2 located within the body portion 6 comprises a hinge 3.

FIG. 3 is a view of the upper jaw 8 as seen from the top and a cross sectional view of the body portion 6 with the sliding block 1 engaged against the inner surface of the distal end of the jaw 5. This is the position required to grasp a suture passed through the upper jaw 8 and has been achieved by the operator pushing against the control member 7 in FIG. 1 which moves the long rod 2 against the sliding block 1. Once this is accomplished, the hinge of the long rod 3 coincides with the hinge of the upper jaw 4 so that when the upper jaw is opened to release the grasped tissue, the position of the sliding block 1 against the inner surface of the distal end of the jaw 5 is maintained so that the suture can be retrieved.

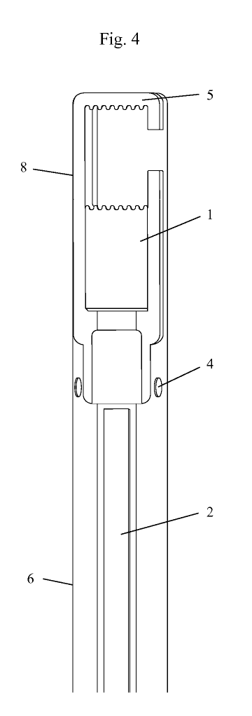

FIG. 4 is a view of the upper jaw 8 as seen from the top and a cross sectional view of the body portion 6 in an alternative embodiment in which the long rod 2 located within the body portion 6 is flexible enough not to require a hinge so that once the long rod 2 is engaged against the sliding block 1, the long rod 2 is flexible enough to bend in order to allow the upper jaw to open.

I claim:

1. a suture passing device comprising:
a. upper and lower jaws which grasp tissue; wherein one of the jaws allows deployment of a suture loaded needle, and the opposing jaw comprises a sliding block;
b. a body portion; and
c. a handle portion.

2. the device mentioned in claim 1 comprising a rod member within the body portion of the suture passing device.

3. the device mentioned in claim 1 comprising a control member near the handle portion of the suture passing device such that engaging the control member transmits force to the rod member which causes the aforementioned sliding block to move and exert compression on a suture for grasping and retrieval.
